(19) United States
(12) Patent Application Publication
(10) Pub. No.: US 2008/0211333 A1
(43) Pub. Date: Sep. 4, 2008

(54) Disk drive compatible new esters for FDB applications with optimized viscosity

(75) Inventors: Raquib Uddin Khan, Pleasanton, CA (US); James H. Smith, Woodside, CA (US)

Correspondence Address: Seagate Technology;c/o DARBY & DARBY P.C., P.O. Box 770, Church Street Station, New York, NY, 10008-0770, US

(73) Assignee: SEAGATE TECHNOLOGY LLC, Scotts Valley, CA (US)

(21) Appl. No.: 11/712,579

(22) Filed: Mar. 1, 2007

(51) Int. Cl. H02K 7/08; C07C 67/30

(52) U.S. Cl. 310/90; 560/203

(57) ABSTRACT

An embodiment of the invention relates to a spindle motor of a magnetic recording storage device, the spindle motor comprising a fluid dynamic bearing comprising a lubricant comprising di-2-ethyl hexyl pimelate, di-2-ethyl hexyl suberate, or combinations thereof.

### RELATED APPLICATION

This application is related to Attorney Docket No. 146712018400, entitled “FREEZING POINT REDUCTION IN FDB BY ENHANCING LUBRICANTS WITH ADDITIVES,” which is incorporated herein by reference.

### FIELD OF THE INVENTION

The present invention relates to a recording media having an advanced lubricant for thin film storage medium and fluid dynamic bearing (FDB) of a disk drive, for example.

### BACKGROUND

Magnetic discs with magnetizable media are used for data storage in most all computer systems. Current magnetic hard disc drives operate with the read-write heads only a few nanometers above the disc surface and at rather high speeds, typically a few meters per second. Because the read-write heads can contact the disc surface during operation, a layer of lubricant is coated on the disc surface to reduce wear and friction.

FIG. 1 shows a disk recording medium and a cross section of a disc showing the difference between longitudinal and perpendicular recording. Even though FIG. 1 shows one side of the non-magnetic disk, magnetic recording layers are sputter deposited on both sides of the non-magnetic aluminum substrate of FIG. 1. Also, even though FIG. 1 shows an aluminum substrate, other embodiments include a substrate made of glass, glass-ceramic, NiP/aluminum, metal alloys, plastic/polymer material, ceramic, glass-polymer, composite materials or other non-magnetic materials.

FIG. 2 shows a fluid dynamic bearing spindle motor. FIG. 2 is a vertical sectional view of a single thrust plate hydrodynamic bearing motor design of a type which is already established in this technology. The basic structure of the motor shown in this figure includes a stationary shaft 10 and a hub 12 supported from a sleeve 13 for rotation around the shaft. The shaft 10 includes a thrust plate 14 at one end, and terminates in a shoulder 16 at the opposite end. The sleeve 13 supports a counterplate 19 at one end, for rotation over the thrust plate 14. The counterplate 19 and thrust plate 14 are separated by a sufficient gap 22 to allow movement of lubricating fluid to lubricate the hydrodynamic bearing through the central hole or reservoir 20, through the gap 22, through the reservoir 26 defined between the end of the thrust plate 14 and an interior surface 27 of the sleeve 13, and between the lower surface 24 of the thrust plate 14 and an upper surface 25 of the sleeve 13, and between an inner surface 28 of the sleeve and the exterior surface 29 of the fixed shaft. The fluid path is completed to reservoir 20 primarily through a central bore 21. In order to promote the flow of fluid over the bearing surfaces which are defined between the thrust plate 14 and the counterplate 19; between the thrust plate 14 and the sleeve 13, and between the shaft 10 and the sleeve 13, typically one of the two opposing surfaces of each such assembly carries sections of grooves as is well known in this technology.

The fluid flow between the bearing surfaces creates hydrodynamic pressure, resulting in stiffness. Circulation of fluid is maintained through central hole 20 of the shaft to the other bearing surfaces by the appropriate designing of geometry and grooving patterns of the baring surfaces. The remainder of the structure of significance which is used to complete the motor design include shaft extension 30 which ends in threaded region 31 which is threaded into a portion of the base 44. A stator 42 cooperates with magnets 40 which are supported from the sleeve 13, with energization of the stator windings 42 causing rotation of the sleeve 18 and the hub 12 about the stationary shaft.

As used in a disc drive motor, this system supports one or more discs 44 for rotation. Because the transducers and disc drives fly at extremely low heights over the surface of the disc, it is essential that there not be wobble or vibration of the hub and disc as it rotates. Moreover, it is also important that should such wobble occur, that there is no touch down between the surfaces of the thrust plate 14 and the opposing surface of the counterplate 19 and sleeve 13. However, as explained above, in a cantilever type bearing such as shown in FIG. 2, where the load carrying surface which is thrust plate 14 is located far from the center point about which any pivoting would occur in the event of vibration or wobble, there is a much greater chance of a touch down or contact between the facing surfaces, which would result in both wear of the surfaces over the long term, and a slow down of the rotational speed of the disc in the short term.

Lubricants in a disc drive are applied on the spindle motor as well as on the disc surface. A lubricant fluid such as oil is typically filled in the bearing space which is created in the gap between the bearing sleeve and the shaft bush of a fluid dynamic bearing. On the other hand, a lubricant is applied to the disc surface by dipping the disc in a bath containing the lubricant or spraying the lubricant to the disc surface.

The lubricant film on the spindle motor or hard discs provides protection to the underlying materials by preventing wear. In addition, it provides protection against corrosion of the underlying materials. Reliability of hard disk drive is depends on the durability of the spindle motor and thin film media. Lubrication plays unquestionably an important role.

There are many common kinds of lubricants presently used in different kinds of fluid dynamic bearing but very few kinds are appropriate for disk drive application. It has been found that disk drive is very sensitive to the type and the amount of chemicals used in different components it is made from. Thus, it is desirable to develop a novel lubricant that would be appropriate for fluid dynamic bearing of disk drive application such that the lubricant exhibits compatibility with disk-head interface.

### SUMMARY OF THE INVENTION

One embodiment of this invention relates to a spindle motor of a,magnetic recording storage device, the spindle motor comprising a fluid dynamic bearing comprising a spindle motor of a magnetic recording storage device, the spindle motor comprising a fluid dynamic bearing comprising a lubricant comprising di-2-ethyl hexyl pimelate. Preferably, the lubricant has a single phase composition. Preferably, the lubricant further comprises a mineral base hydro carbon, synthetic hydrocarbon containing compound, or an ester selected from the group consisting of diester, monoester, simple ester, compound ester and combinations thereof. Preferably, the lubricant further comprises an additive. Preferably, the lubricant has a viscosity in the range of 4 to 20 cst @ 40° C.

Another embodiment of the invention relates to a spindle motor of a magnetic recording storage device, the spindle motor comprising a fluid dynamic bearing comprising a lubricant comprising di-2-ethyl hexyl suberate. Preferably, the lubricant has a single phase composition.

Another embodiment relates to a method of manufacturing di-2-ethyl hexyl pimelate comprising reacting pimelic acid and 2-ethyl hexyl alcohol.

Yet another embodiment of the invention relates to a method of manufacturing di-2-ethyl hexyl suberate comprising reacting suberic acid and 2-ethyl hexyl alcohol.

Yet another embodiment of the invention relates to a method of manufacturing polyol ester comprising reacting monocarboxylic acid and polyol alcohol.

Additional advantages of this invention will become readily apparent to those skilled in this art from the following detailed description, wherein only the preferred embodiments of this invention is shown and described, simply by way of illustration of the best mode contemplated for carrying out this invention. As will be realized, this invention a property of other and different embodiments, and its details are capable of modifications in various obvious respects, all without departing from this invention. Accordingly, the drawings and description are to be regarded as illustrative in nature and not as restrictive.

### BRIEF DESCRIPTION OF THE DRAWINGS

The present invention will be better understood by reference to the Detailed Description of the Invention when taken together with the attached drawings, wherein:

FIG. 1 shows a magnetic recording medium.

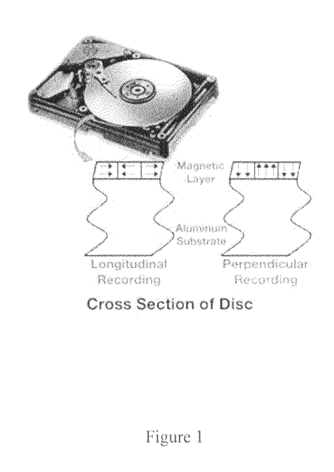

FIG. 2 shows a fluid dynamic bearing spindle motor.

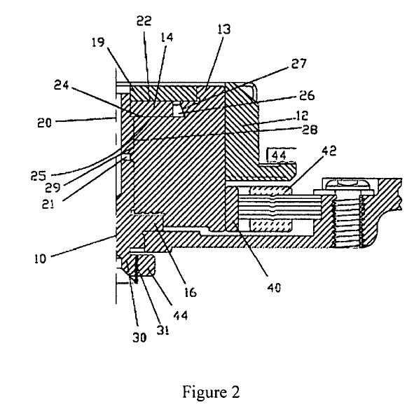

### DETAILED DESCRIPTION OF THE INVENTION

As used in the specification and claims, the singular forms “a”, “an” and “the” include plural references unless the context clearly dictates otherwise.

The invention is directed to a lubricant for a disc drive and is referred in the specification to as a “lube.” Lubricants typically are liquid and contain molecular weight components that range from few hundred Daltons to several thousand Daltons.

While doing research in lubricants for disk drive spindle motors, the inventor encountered many lubricants which might have all the required properties to be applied in a spindle motor having a FDB but they were marginally acceptable. Presently used lubricants include Di-Octyl Sebacate, Di octyl Azelate, Di-octyl adipate, which are the reaction products of 2-Ethyl-1-hexanol (Isooctyl alcohol) and the dibasic acids of C10 (10 carbon), C9 and C6 respectively (e.g., sebacic acid, azelaic acid and adipic acid). In this type of ester, higher the number of carbon atoms in the molecule of the lubricant, usually higher is the viscosity and lower is the evaporation. But based on the sensitivity of the FDB application, these fluids are often not sufficient to generate any intermediate viscosity with optimized evaporation as mixing principle works for viscosity optimization but it may not work for evaporation optimization. In most cases, higher volatile esters try to evaporate much faster than the low volatile components if the selection of the chemicals is not done carefully.

Hence, an embodiment of the invention relates to novel lubricants that can avoid this situation. The novel lubricants are preferably single phase, single component ester-containing compounds with intermediate viscosity and optimized evaporation. The embodiments of the novel lubricants of the present invention contain one or more compounds that are preferably made from the reaction with C8 and C7 or similar type dibasic acids with Iso-octyl alcohol or similar compounds. The reaction products, e.g., di-2-ethyl hexyl suberate and di-2-ethyl hexyl pimelate, have intermediate viscosity and optimized evaporation with all other good properties of the presently used multiple ester-containing compounds in the lubricants of FDB for disk drive application.

Another embodiment of the invention relates to optimize the viscosity for a large change in temperature. The lower the change in viscosity (higher VI number), better it is for application. Hence, the present invention directs toward using the reactants carefully by selecting the proper alcohol type and acids. As an example, n-octyl, isooctyl, 2-Ethylhexyl alcohol—all have 8 carbons and when reacted with diacid like adipic acid—all create a dioctyl adipate but the properties are entirely different with same molecular weight. The n-octyl adipate will result in higher viscosity product and better viscosity index (low change in viscosity with temperature) but has to sacrifice in freezing point (higher freezing point compare to isooctyl adipate). Similarly, the novel invention can optimize the lubricant properties by reacting different types of alcohol-linear versus branched with different diacids like pimelic, suberic or sebacic acid.

In accordance with the present invention, the lubrication fluid could comprise a base fluid and optionally at least one additive. Preferred base fluids include perfluoropolyethers (PFPEs), esters, synthetic hydrocarbons, and highly refined mineral hydrocarbons. Most preferred base fluids include di-2-ethyl hexyl suberate and di-2-ethyl hexyl pimelate. These base fluids can also be blended in a variety of combinations.

The additive could be selected, for example, so as to change the surface tension value at the gas-lubricant interface of the lubrication fluid. The additive could be at least partially soluble in the base fluid and have a low surface tension value compared to the base fluid. Additives that can be used with the present invention include polysiloxanes (silicones), polyacrelates, organic copolymars, and fluorocarbon compounds, such as PFPEs. Specific PFPEs that can be used with the present invention include FOMBLIN Z-DOL and FOMBLIN AM-2000, both commercially available from Ausimont, located in Morristown N.J. Z-DOL is a random copolymer of perfluorinated ethylene oxide and perfluorinated methylene oxide. AM-2000 is a difunctional aromatic terminated perfluoropolyether. Another additive that can be used with the present invention is VANLUBE DF 283, commercially available from RT Vanderbilt, located in Norwalk, Conn.

The surface tension of the lubrication fluid could be less than 35 dynes/cm, preferably in the range between 12 and 35 dynes/cm. For example, the surface tension of a typical base fluid (e.g., ester oil) is between 28 and 35 dynes/cm. The additive could cause the lubrication fluid to preferably have as surface tension lower than that of the base fluid alone. The additive could comprise between 0.02% and 2.0% by volume of the lubrication fluid.

The additives might also be added to improve other properties like oxidation resistant, wear resistance, corrosion protection etc.

It is desirable that the lubricant has a relatively narrow molecular weight distribution of molecular components. In practice, the narrower the distribution the easier it will be to maintain a steady-state concentration of one or more components in the vapor. For example, if the highest and lowest molecular weight components in the polymer have very similar molecular weights, their vapor pressures will also be very similar. On the other hand, if the molecular weights (vapor pressures) are dramatically different heating of the lubricant will require much greater temperature and process control for a steady state concentration to be maintained. The lubricant used in the invention should have an M_{w}/Mₙ ratio between 1 and 1.6, preferably between 1 and 1.3, more preferably between 1and 1.2.

Diesters of interest can be synthesized from either alcohols and diacids or monoacids and diols:

2ROH+HO₂C(CH2)ₙCO₂H [symbol] RO₂C(CH2)ₙCO₂R

CO₂H+HO(CH₂)ₙOH [symbol] RCO2(CH₂)ₙO₂R

For the alcohols in the first reaction, any alcohol can be used, preferably with 4 to 10 carbons. For the diacid, n can be between 4 and 12. For the second reaction, the mono acids may include heptanoic acid, octanoic acid, 2-ethylhexanoic acid, nonanoic acid, decanoic acid, and other branched chain isomers. The diols, HO(CH₂)ₙOH, may have n from 4 to 12. Also, the branched chain diol neopentyl glycol can be used. In the same way, polyesters from polyols such as trimethyolpropanol (TMP), pentaerythritol, and other polyols can be used. As the number of alcohol groups increases, the chain length of the acid must decrease to keep the viscosity in the desired range.

### EXAMPLES

The following ester-containing lubricants were prepared by the reaction of an acid a dioctyl alcohol:

| Acid | Dioctyl alcohol | Ester |
|---|---|---|
| Adipic acid (6 carbon) | 2-ethyl hexyl alcohol (8 carbon) | Di-2-ethyl |
| | | hexyl adipate |
| Pemelic acid (7 carbon) | 2-ethyl hexyl alcohol (8 carbon) | Di-2-ethyl |
| | | hexyl |
| | | pimelate |
| Phthalic acid (8 carbon) | 2-ethyl hexyl alcohol (8 carbon) | Di-octyl |
| | | phthlate |
| Suberic acid (8 carbon) | 2-ethyl hexyl alcohol (8 carbon) | Di-2-ethyl |
| | | hexyl |
| | | suberate |
| Azelaic acid (9 carbon) | 2-ethyl hexyl alcohol (8 carbon) | Di-octyl |
| | | azelate |
| Sebacic acid (10 carbon) | 2-ethyl hexyl alcohol (8 carbon) | Di-octyl |
| | | sebacate |

The method of synthesis of the above esters was as follows.

### Synthesis of Di-2-Ethylhexyl Pimelate

The following chemicals listed in Table 1 were added to a 250 mL round bottom flask:

| | CAS | | | |
|---|---|---|---|---|
| Chemical | Number | FW | Weight (g) | Moles |
| Pimelic Acid | 111-16-0 | 160 | 48.00 | 0.300 |
| 2-Ethyl-1-hexanol | 104-76-7 | 130 | 85.90 | 0.661 |
| Toluene | 108-88-3 | 92 | 25.00 | 0.272 |
| Sulfuric Acid (95%) | 7664-93-9 | 98 | 0.15 | 0.002 |
| Activated carbon | 7440-44-0 | 12 | 1.50 | 0.125 |

A small stirring bar and boiling chips were also added to the flask. A Dean-Stark trap and condenser were then connected to the flask. The mixture was refluxed for about 2 hours and the water from the esterification reaction (about 10 mL) was collected in the trap and drained. Most of the toluene was removed by distillation at atmospheric pressure. The reaction mixture was cooled to room temperature and about 1 g of potassium carbonate was added to neutralize the sulfuric acid and any unreacted organic acids. Then about 1 g of anhydrous sodium sulfate was added to the mixture to remove any residual water. The mixture was filtered and the clear, colorless ester was retained. The remaining toluene and other low molecular weight impurities were distilled from the reaction product under vacuum to yield the pure diester. The purity and identity of the diester was conformed by gas chromatography-mass spectroscopy (GC-MS). This same method was used to synthesize diesters of 1,6-hexanediol, 1,9-nonanediol, and neopentyl glycol in good yields and purity.

It was found that even though di-octyl phthalate is made by the reaction of an acid (phthalic acid) having 8 carbon atoms with a dioctyl alcohol, di-octyl phthalate has a totally different structure than that of di-2-ethyl hexyl suberate, which is also made by the reaction of an acid (suberic acid) having 8 carbon atoms with a dioctyl alcohol. This is because phthalic acid is a ring compound. Hence the viscosity of di-octyl phthalate is very high.

It was also found that a mixture of adipic acid (6 carbon) and sebacic acid (10 carbon) reacted with dioctyl alcohol could produce a mixed ester of di-2-ethyl hexyl adipate and di-octyl sebacate having a balanced viscosity suitable for fluid dynamic bearing for disk drive application. However, such as mixed ester would be dominated by the evaporation of the volatile part of the mixed ester, which is di-2-ethyl hexyl adipate.

On the other hand, di-2-ethyl hexyl pimelate and di-2-ethyl hexyl suberate, which have an intermediate molecular weight, have a viscosity appropriate for fluid dynamic bearing for disk drive application and much lower evaporation rate as compared to that of di-2-ethyl hexyl adipate.

In this application, the word “containing” means that a material comprises the elements or compounds before the word “containing” but the material could still include other elements and compounds. This application discloses several numerical ranges in the text and figures. The numerical ranges disclosed inherently support any range or value within the disclosed numerical ranges even though a precise range limitation is not stated verbatim in the specification because this invention can be practiced throughout the disclosed numerical ranges.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. Finally, the entire disclosure of the patents and publications referred in this application are hereby incorporated herein by reference.

What is claimed is:

1. A spindle motor of a magnetic recording storage device, the spindle motor comprising a fluid dynamic bearing comprising a lubricant comprising di-2-ethyl hexyl pimelate.

2. The spindle motor of magnetic recording storage device of claim 1, wherein the lubricant has a single phase composition.

3. The spindle motor of magnetic recording storage device of claim 1, wherein the lubricant further comprises a mineral base hydro carbon, synthetic hydrocarbon containing compound, or an ester selected from the group consisting of diester, polyol ester, monoester, simple ester, compound ester and combinations thereof.

4. The spindle motor of magnetic recording storage device of claim 3, wherein the lubricant further comprises one or more additives.

5. The spindle motor of magnetic storage device of claim 1, wherein the lubricant has a viscosity in the range of 4 to 20 cst @ 40° C.

6. A spindle motor of a magnetic recording storage device, the spindle motor comprising a fluid dynamic bearing comprising a lubricant comprising di-2-ethyl hexyl suberate.

7. The spindle motor of magnetic recording storage device of claim 6, wherein the lubricant has a single phase composition.

8. The spindle motor of magnetic recording storage device of claim 6, wherein the lubricant further comprises a mineral base hydro carbon, synthetic hydrocarbon containing compound, or an ester selected from the group consisting of diester, polyol ester, monoester, simple ester, compound ester and combinations thereof.

9. The spindle motor of magnetic recording storage device of claim 8, wherein the lubricant further comprises one or more additives.

10. The spindle motor of magnetic storage device of claim 6, wherein the lubricant has a viscosity in the range of 4 to 20 cst @ 40° C.

11. The spindle motor of magnetic recording storage device of claim 6, wherein the lubricant further comprises di-2-ethyl hexyl pimelate.

12. A method of manufacturing di-2-ethyl hexyl pimelate comprising reacting pimelic acid and 2-ethyl hexyl alcohol.

13. A method of manufacturing di-2-ethyl hexyl suberate comprising reacting suberic acid and 2-ethyl hexyl alcohol.

14. A method of manufacturing a viscosity lubricant for a range of temperature comprising obtaining a linear and or branched alcohol with a diacid and reacting the linear and or branched alcohol with the diacid.
